(19) United States
(12) Patent Application Publication
(10) Pub. No.: US 2008/0234572 A1
(43) Pub. Date: Sep. 25, 2008

(54) FIDUCIAL MARKER WITH ABSORBABLE CONNECTING SLEEVE AND ABSORBABLE SPACER FOR IMAGING LOCALIZATION

(75) Inventor: Andrew Douglas Jones, Lake Steven, WA (US)

Correspondence Address: CAESAR, RIVISE, BERNSTEIN,;COHEN & POKOTILOW, LTD., 11TH FLOOR, SEVEN PENN CENTER, 1635 MARKET STREET, PHILADELPHIA, PA, 19103-2212, US

(73) Assignee: CIVCO MEDICAL INSTRUMENTS CO., INC., Kalona, IA (US)

(21) Appl. No.: 11/690,470

(22) Filed: Mar. 23, 2007

(51) Int. Cl. A61B 5/05

(52) U.S. Cl. 600/426

(57) ABSTRACT

A fiducial marker for use in the body of a living being to enable the localization of targeted internal tissue. The implant is in the form of an elongated, e.g., rod-like, spacer located between pair of end members, e.g., gold cylinders, and held together by an elongated sleeve, e.g., a heat-shrinkable sleeve. When so connected portions of the end members are exposed, i.e., extend beyond the ends of the sleeve. The outer surface of the exposed portions of each of the end members is textured, e.g., grooved, to provide resistance to migration when the marker is implanted. The spacer and the sleeve are each be formed of an absorbable material.

### FIELD OF THE INVENTION

This invention relates generally to fiducial markers for implantation into the body of a living being and more particularly to fiducial markers which facilitate radiographic, ultrasound or magnetic resonance imaging of such markers and methods of using the same.

### BACKGROUND OF THE INVENTION

The clinical success of focused, dose-delivery procedures, such as intensity modulated radiation therapy (IMRT) and conformal radiation therapy (CRT), is based on the accuracy of target identification and precise patient positioning. Image-guided localization is best achieved by utilizing implanted fiducial markers. In particular, such markers allow for accurate tumor localization using a variety of visualization techniques, and help these focal radiotherapy procedures by enabling real-time targeting of tumors. In addition higher doses of radiation can be targeted to the tumor and its periphery. As the trend to higher doses, smaller number of fractions, and tighter margins becomes an important part of radiotherapy, better verifiable localization is a necessity. Image-guided localization provides this type of real-time, high-precision localization. Thus, use of such markers has been deemed imperative in situations where the targeted tissue moves with respect to external marks (e.g., tattoos). In particular, prostate, liver and other such internal organs can be much more accurately targeted using implanted markers. In cases where the targeted tissue moves in an identical fashion with respect to the adjacent bony anatomy it is possible to utilize the bony anatomy as the registration points.

The Assignee of the subject invention, CIVCO Medical Solutions, of Kalona, Iowa, offers image-guided patient localization systems for localizing targeted tissue, e.g., soft tissue, such as prostate tumors, etc., under the trademark ACCULOC®. The ACCULOC® system makes use of fiducial markers in conjunction with ISOLOC™ software and electronic portal imaging (EPID), computed radiography, or standard port films to accurately locate the tissue in which the fiducial markers are implanted and thereby provide precise patient (e.g., couch) movement to achieve desired target alignment. In particular, the ISOLOC™ software algorithms provide for high-precision localization based on unique anatomic points. One of the algorithms allows the user to click on two unique points from an anterior view and a completely separate set of two points from a lateral projection. These four points are back-projected into the original 3D data-set used for planning. The program resolves the location of these points at the time of treatment and provides the couch shifts to precisely align the target. Other algorithms include single projection localization, image matching, and automated marker detection. Thus, using the ACCULOC® system markers in conjunction with the ISOLOC™ software one can easily register the target location with the treatment beam for precision dose delivery.

The markers presently sold as part of the ACCULOC® system are made of gold and are cylindrical in shape and 3 mm in length, but are available in three different diameters: 0.9 mm, 1.2 mm and 1.6 mm. The shape and size of these markers enables them to be easily inserted using a needle under ultrasound or CT guidance. The markers may be pre-loaded in needles as single markers, or as marker pairs spaced at 10 or 15 mm apart. In order to prevent migration the surface of each of the ACCULOC® soft tissue markers is specially treated, e.g., knurled.

In U.S. patent application Ser. No. 11/422,872, filed on Jun. 7, 2006, entitled Integrated Real-Time Localization Platform Using Kilovoltage X-Rays, which is assigned to the same assignee as the subject invention and whose disclosure is incorporated by reference herein, there is disclosed and claimed a system and method for patient organ localization using fiducial markers. That system includes a kilovoltage source, a real-time marker detector and a robotic table top. The application also discloses apparatus for organ motion simulation including a motion platform controller, motion control actuators and graphical user interface for controlling the motion platform controller.

Other fiducial markers are currently available from the following companies. Best Industries, W. E. Mowrey Company, Alpha-Omega Services, Inc., and RadioMed Corporation. For example, Best Industries offers a similar marker to the markers of the ACCULOC® system, except that the Best Industries marker is not knurled. W. E. Mowrey Company, has provided markers in the form of cut-up gold wire sections of approximately 1.2×3.0 mm. Alpha-Omega Services, Inc. sells gold markers. RadioMed Corporation sells linear fiducial soft tissue markers used to localize organs, tumors and tumor beds for image-guided radiation therapy under the trade designation VISICOIL. The VISICOIL markers are in the form of an elongated helical gold coils.

The prior art also includes patents disclosing fiducial markers and method of using the same, such as U.S. Pat. Nos. 5,397,329 (Allen) and 6,694,168 (Traxel et al.).

In U.S. patent application Ser. No. 11/461,813, filed on Aug. 2, 2006, entitled Fiducial Marker For Imaging Localization And Method Of Using the Same, of which I am a co-inventor and which is assigned to the same assignee as this invention and whose disclosure is incorporated by reference herein, there is disclosed a fiducial marker that overcomes many of the disadvantages of the prior art. In particular, that fiducial marker comprises an elongated rod-like, central section interconnecting a pair of end sections. Each of the end sections is of a bulbous, e.g., spherical, shape that is readily imaged by a first type of imaging, e.g., X-ray imaging, ultrasonic imaging or magnetic resonance imaging. The central section holds the two end sections together at a desired spacing and is formed of a material, e.g., titanium, that is not readily imaged by the first type of imaging, whereupon when the implant is implanted in the targeted tissue and exposed to the first type of imaging the bulbous ends of the implant can be readily discerned in the targeted tissue, with the central section being less discernable in the targeted tissue. The fiducial implant is arranged to be inserted via a needle or cannula into targeted tissue in the body of the living being, whereupon when implanted in the targeted tissue and exposed to the first type of imaging, the bulbous end sections of the implant can be readily discerned in the targeted tissue.

While the fiducial markers of that invention are suitable for their intended purposes, they leave something to be desired from the standpoint of ready assembly.

Best Medical International, Inc. of Springfield Virginia has advertised fiducial markers which appear to enable their ready assembly. Such markers make use of two gold cylinders which are spaced from each other by a spacer element and held in that spaced apart position by use of a sleeve which extends over the entire length of the gold cylinders and the interposed spacer. While such markers appear suitable for their intended purposes, they may not be as resistant to migration once implanted as would be desired.

Thus, a need exists for a fiducial marker that can be readily assembled, yet which will be very resistant to migration once implanted, and still provide excellent imaging capabilities. The subject invention addresses that need.

### SUMMARY OF THE INVENTION

In accordance with one aspect of this invention there is provided a fiducial marker for implantation in the body of a living being to locate targeted tissue when imaged by a first type of imaging. The marker comprises an elongated spacer, a pair of end members and a sleeve. The spacer has a pair of ends. Each of the end members has an outer surface, at least a portion of which is textured to be somewhat roughened, e.g., grooved. Moreover, each of the end members is formed of a material, e.g., gold, that is readily imaged by a first type of imaging. The sleeve is an elongated tube-like member, e.g., a heat shrinkable tube, having a pair of ends and a central passageway extending through it. The spacer is located within the central passageway of the sleeve. Each of the end members includes a respective inner end portion located immediately adjacent a respective one of the ends of the spacer and secured (e.g., when the heat shrinkable tube is shrunk each of the end members is held by frictional engagement) at its position within the passageway, whereupon a portion of the textured surface of each of the end members is exposed to extend outside of the sleeve. Accordingly, when the marker of this invention is implanted in the targeted tissue it is resistant to migration and its end members can be readily discerned in the targeted tissue by the first type of imaging.

In accordance with another aspect of this invention the spacer is constructed so that it is not readily imaged by the first type of imaging, whereupon when the fiducial marker is implanted in the targeted tissue and exposed to the first type of imaging the end members can be readily discerned in the targeted tissue, with the spacer being less discernable in the targeted tissue. The spacer and the sleeve are preferably constructed of absorbable materials.

In accordance with still another aspect of this invention the size and shape of the end members and the interposed spacer is such that when the heat-shrinkable sleeve is shrunk the resultant fiducial marker has an over-all dumbbell-like shape, thereby rendering it further resistant to migration once implanted.

### DESCRIPTION OF THE DRAWING

FIG. 1 is an isometric view of one exemplary assembled fiducial marker constructed in accordance with this invention and which is made up of a pair of end members, an interposed spacer and a heat-shrinkable sleeve, which when shrunk as shown holds all of the components together as shown in this figure;

FIG. 2 is a view like that of FIG. 1 but showing the various components making up the marker in their assembled state immediately prior to the heat-shrinkable sleeve being heated to shrink;

FIG. 3 is a view like that of FIG. 2, but showing the internal portions of the marker by means of broken lines; and

FIG. 4 is an isometric view of the spacer forming a portion of the fiducial marker of FIG. 1.

### DETAILED DESCRIPTION OF THE PREFERRED EMBODIMENT

As will be appreciated by those skilled in the art, there are several key considerations with respect to implanted fiducial markers. In particular, the markers must be clearly visible in the radiotherapy planning image study, typically accomplished by computer tomography (CT). The markers must also be clearly visible with the treatment machine imager, whether that be electronic portal imager, x-ray film or computerized radiograph. In addition, the markers must not disrupt or distort any of the aforementioned imaging modalities. Since the markers are implanted in tissue or bone they must be formed biocompatible materials. Lastly, and perhaps most importantly, the markers must remain (relatively) stable with respect to the target location and to each other from the time of treatment planning imaging study and there after until treatment is completed in order to ensure that the target tissue can be precisely located with respect to all three dimensional directions.

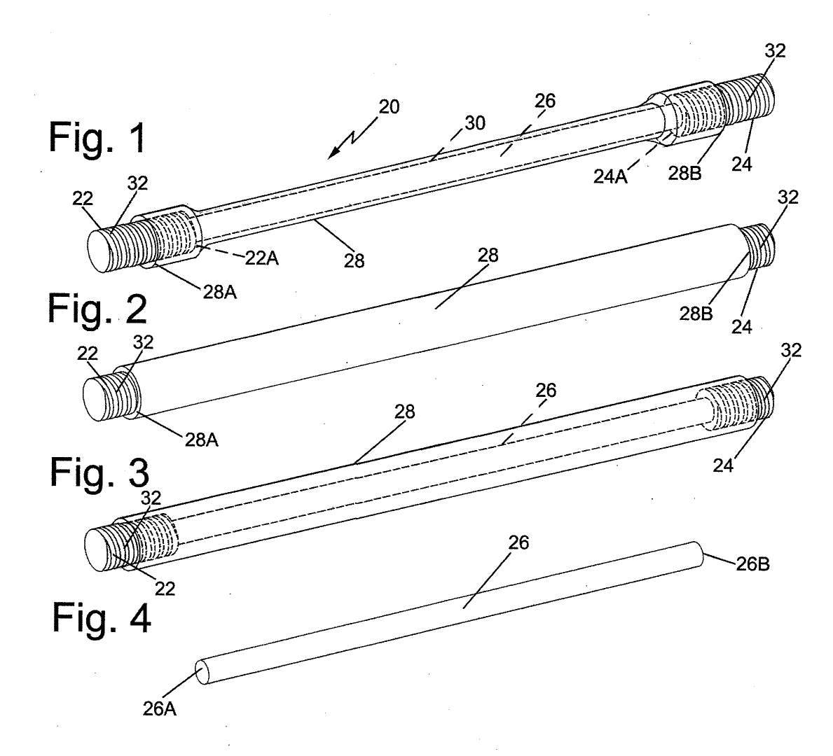

Referring now to the various figures of the drawing wherein like reference characters refer to like parts, there is shown at 20 in FIG. 1 a fiducial marker that meets the aforementioned design criteria for such devices. Thus, as will be described in detail hereinafter, the subject fiducial markers of this invention achieve all of the above design criteria. In particular, the fiducial markers of this invention are made up of two end members 22 and 24, each of which can be imaged by a first type of energy, e.g., radiographically imaged by X-rays. The end members are spaced from each other by a desired, fixed and known distance via an interposed spacer 26 to facilitate targeting of the tissue in which the marker 20 is implanted. The end members 22 and 24 and the interposed spacer 26 are held together by a sleeve 28, which in this exemplary preferred embodiment is heat-shrinkable, so that when the marker is assembled portions of the end members 22 and 24 (to be described shortly) are exposed. These exposed portions have a textured, e.g, roughened, surface to inhibit migration of the marker when it is implanted in the targeted tissue. Moreover, the finished marker has an over-all, dumbbell like shape, similar to the marker of my aforementioned copending patent application Ser. No. 11/461,813, which shape also renders the marker 20 resistant to migration. Thus, the fiducial markers of this invention can be used in any procedure and with any system(s) heretofore making use of fiducial markers, e.g., in systems and with methods like those disclosed in the aforementioned copending application Ser. No. 11/422,872.

In accordance with a preferred embodiment of this invention for use by X-ray imaging each of the end members 22 and 24 is of a cylindrical shape and formed of gold. That construction is merely exemplary. Thus for example, the end members 22 and 24 could be of other shapes having a circular cross section, e.g., a sphere, an ovoid, etc., so that each has a respective maximum outer diameter. Moreover, other materials than gold that have been used in the prior art can be used for the end members 22 and 24. In the embodiment of the fiducial marker 20 shown in the drawing, the end members 22 and 24 are identically shaped and sized cylindrical gold members, e.g., the outer diameter of each being constant throughout its length and is within the range of approximately 0.2 mm to 2.0 mm, depending upon the application to which to marker 20 will be put. Each end member 22 and 24 has a length in the range of approximately 1.0 mm to 12.0 mm, again depending upon the application to which to marker 20 will be put. As best seen in FIG. 3, the end members 22 and 24 include inner end surfaces 22A and 24A.

As best seen in FIG. 4, the spacer 26 is an elongated, linear, rod-like member that includes a pair of ends 26A and 26B, and is formed of a material (to be described later) that is relatively stiff to maintain the distance between the end members 22 and 24. The spacer 26 is of an outer diameter that is less than the outer diameter of the end members 22 and 24 so that when the heat As best seen in FIG. 3, the end member 22 is disposed immediately adjacent the end 26A of the spacer 26 so that its end surface 22A abuts the end 26A of the spacer. In a similar manner, the end member 24 is disposed immediately adjacent the end 26B of the spacer 26 so that its end surface 24A abuts the end 26B of the spacer. The spacer 26 is of circular cross-section, but can be of other shapes as well. The outer diameter of the spacer is preferably less than the diameter of the end members 22 and 24 so that when the sleeve 28 is shrunk the resulting fiducial marker has an over-all dumbbell-like shape. If it is desired that the marker not have a dumbbell like shape, the outer diameter of the spacer may be the same as the outer diameter of the end members 22 and 24. In any case, the length of the spacer is selected so that when it is interposed between the end members 22 and 24, with the end members abutting its ends 26A and 26B, the spacing between the respective centers of the two end members is within the range of 5 mm to 50 mm, with a preferred spacing of approximately 20 mm.

The sleeve 28 is an elongated, linear, tube-like member having a pair of ends 28A and 28B and a central passageway 30 extending through the sleeve between those ends. The passageway 30 is arranged to receive the entire spacer 26 and an inner end portion of each of the end members 22 and 24, i.e., portions of the end members contiguous with the end surfaces 22A and 24A, respectively. Thus, the length of the sleeve 28 and the inner diameter of its passageway 30 is dependent on the sizes of the spacer 26 and the end members 22 and 24. As mentioned above, in accordance with a preferred embodiment of this invention sleeve 26 is formed of a heat-shrinkable material. In particular, in its un-shrunk state the sleeve is of a slightly larger internal diameter than the maximum external diameter of the end members 22 and 24. For example, for some exemplary embodiments of this invention the internal diameter of the passageway 30 in the sleeve is approximately 0.89 mm. Moreover, the sleeve is of a length that is longer than the length of the spacer, but less than the combined length of the end members and the spacer. Accordingly, the un-shrunk sleeve 28 can easily receive in its passageway 30 the spacer and the end members, with the end member abutting respective ends of the spacer. This action can be accomplished by either introducing those components within the interior of un-shrunk sleeve or by sliding the un-shrunk sleeve over those components. In either case once all of the fiducial marker's components are in their desired positions with respect to one another, like shown in FIGS. 2 and 3, the heat-shrinkable sleeve 28 can be exposed to heat, e.g., heated with a heat gun or introduced into a heating oven, whereupon the sleeve shrinks to the state like shown in FIG. 1, so that its ends 28A and 28B are located inward of the free ends of the end members 22 and 24, thereby exposing significant portions of the textured outer surface 32 of those end members. The shrinkage of the sleeve on the respective portions of the end members results in a good (e.g., tight) frictional engagement between the sleeve and the abutting portions of the end members, thereby fixing them in position with respect to each other.

It should be pointed out at this juncture that in lieu of the use of a heat shrinkable sleeve, the sleeve may be of a fixed size, whereupon the inner diameter of the passageway 30 in the sleeve and length of the sleeve must be chosen so that the end members 22 and 24, with the interposed spacer, can be readily located (e.g., placed) within that passageway and then secured in place. To that end, if a dumbbell shaped marker is desired, it is contemplated that the inner diameter of the passageway 30 at the middle of the sleeve be equal to or just slightly larger than the outer diameter of the spacer 26 to accommodate the spacer therein while inner diameter of the passageway 30 at the ends 28A and 28B is equal to or just slightly less than the outer diameter of the end members 22 and 24 so that they can be secured within the passageway 30 of the sleeve 28 by frictional engagement. Alternatively, the internal diameter of the sleeve at the ends 28A and 28B may be larger so that the end members 22 and 24 can be secured therein by means other than frictional engagement, e.g., by means of an adhesive or some other securement component.

In order to keep the outer diameter of the fiducial marker 20 to a minimum, e.g., to keep it to a dimension not substantially greater than the outer diameter of the end members 22 and 24, thereby facilitating the implantation of the marker into the targeted tissue by any conventional means, e.g., a needle (not shown), the sleeve 28 when shrunk (if a heat-shrinkable sleeve is used) is preferably relatively thin walled, e.g., 0.038 mm and 0.051 mm.

As will be appreciated by those skilled in the art, in order to ensure precise localization of the targeted tissue it is of considerable importance that the distance between the two end portions 22 and 24 of the fiducial marker 20 be fixed and maintained at that fixed, consistent distance from each other when the marker is implanted (the fixed distance can be stored in the software for verification purposes). Accordingly, as mentioned above, the spacer 26 is constructed to be substantially rigid so that it doesn't flex or otherwise deform, which action could change the spacing between the end members 22 and 24 of the marker. As a further means of ensuring that the central portion of the fiducial marker 20 cannot bend, it is desirable (although not mandatory) that the sleeve 28, when shrunk, be somewhat stiff. By making use of such a construction the rigidity of the shrunk sleeve 28 can aid the rigid spacer 26 in deterring bending of the resulting fiducial marker.

As mentioned earlier, the exterior surfaces of the end member 22 and 24 are textured or roughened. In particular, as best seen in FIG. 3, the outer cylindrical surface of each of those members is grooved, e.g., includes a spiral groove 32 extending about the entire length of that surface. Alternatively, the grooves may be configured to form a knurled texture. Other textures can be used as well, so long as the resultant surface is somewhat roughened. The grooved or roughened outer surface of each of the end members 22 and 24 aids in their frictional engagement within the passageway 30 of the sleeve 28. Moreover, and more significantly, the roughened outer surface of the end members 22 and 24 serves as a means for ensuring that the fiducial marker 20 is resistant to migration when implanted. To that end, as should be apparent from the exemplary dimensions of the components of the fiducial marker as described above, when the fiducial marker 20 of this invention is assembled the free ends of the end members 22 and 24, i.e., the portions of the end members disposed opposite the ends 22A and 24A, respectively, are exposed, i.e., lay beyond the ends 28A and 28B, respectively, of the sleeve 28.

Assembly of the fiducial marker is a very simple matter that can be accomplished in several ways. For example, the spacer 26 can be inserted into an un-shrunk, heat-shrinkable sleeve 28 so that it is located at the center thereof. The end members 22 and 24 can then be inserted through the open ends 28A and 28B, respectively, of the sleeve into its passageway 30 until their inner ends 22A and 24A abut the ends 26A and 26B, respectively, of the spacer as shown in FIG. 3. The heat-shrinkable sleeve 28 can then be heated by any suitable means (not shown), such as a heat gun, heat tunnel, etc., whereupon it will shrink to the state shown in FIG. 1 so that the free end portions of the end members 22 and 24 extend beyond the ends 28A and 28B of the now shrunk sleeve 28.

In accordance with a preferred embodiment of this invention the spacer and the sleeve are each arranged to be absorbed by the body so that they eventually disappear, leaving the two end members within the targeted tissue at the desired spacing with respect to each other. However, even before the sleeve and the spacer are absorbed, the end members 22 and 24 will still be significantly more discernable through imaging than the sleeve and/or the spacer by virtue of the materials from which they are made. Accordingly, when the fiducial marker 20 is in position in the tissue to be localized, only the end portions 22 and 24 will be visible. This action effectively create a pair of points that can be used with the associated software, like the ISOLOC™ software, to help localize the marker. For most applications two fiducial markers will be implanted, thereby creating four discrete localizable points. The software may only need to utilize three of those points to localize the targeted tissue.

The material forming the absorbable spacer 26 can be any material suitable for that purpose. One particularly useful material is a dyed 90/10 PGA PLLA copolymer that is available from CP Medical Corporation of Portland, Oreg. under the trade designation Biospacer 910. The material forming the heat-shrinkable sleeve 28 can be any material suitable for that purpose. In addition it is preferred that the material forming the sleeve also be absorbable. One particularly useful material to meet both of those criteria is an undyed 20/80 PLLA/PGA copolymer also available from CP Medical under the trade designation ISO 20/80. Other suitable heat-shrinkable, absorbable materials can be used for the sleeve.

The markers of this invention are arranged to be loaded into a needle or cannula (not shown), for delivery into the tissue to be targeted, e.g., the prostate. To that end one or more markers 20 is disposed within the hollow central lumen in the needle just proximally of a removable plug (not shown). The plug is formed of a biologically inert sterile wax and serves to hold the marker(s) in place in the lumen between it and a pusher rod. The sharpened distal end of the needle is directed into the targeted tissue and the pusher rod actuated to push the plug and the fiducial marker out of the lumen and thereby deposit the plug and marker in the targeted tissue. If the needle contains a second marker the needle can be retracted and then introduced into a second site in the targeted tissue to deposit the second marker thereat. If the needle only contains one marker 20 another needle will be required to deposit the second marker 20 in the targeted tissue. Thus, using the subject invention only two “needle sticks” will be necessary to produce four discernable points for localizing the targeted tissue (although only three of such points may be necessary with the localization software), whereas prior art systems for localizing the targeted tissue would require at a minimum three needle sticks. This feature offers a considerable advantage insofar as patient comfort is concerned. Moreover, as noted above the roughened surfaces of the exposed end members of the markers, particularly when combined with the over-all dumbbell-like shape of the marker results in a fiducial marker having excellent resistance to migration while providing plural discernable imaging points which remain at a consistent fixed distance from each other.

Without further elaboration the foregoing will so fully illustrate my invention that others may, by applying current or future knowledge, adopt the same for use under various conditions of service.

I claim:

1. A fiducial marker for implantation in the body of a living being to locate targeted tissue when imaged by a first type of imaging, said marker comprising an elongated spacer, a pair of end members and a sleeve, said spacer having a pair of ends, each of said end members having an outer surface at least a portion of which is textured to be somewhat roughened, each of said end members being formed of a material that is readily imaged by a first type of imaging, said sleeve being an elongated tube-like member having a pair of ends and a central passageway extending through the length of said sleeve, said spacer being located within said central passageway, each of said end members including a respective inner end portion located immediately adjacent a respective one of said ends of said spacer and secured at said positions within said passageway, whereupon a portion of said textured surface of each of said end members is exposed to extend outside of said sleeve, so that when said marker is implanted in the targeted tissue the marker is resistant to migration and said end members can be readily discerned in the targeted tissue by the first type of imaging.

2. The fiducial marker of claim 1 wherein said spacer is not readily imaged by the first type of imaging, whereupon when said fiducial marker is implanted in the targeted tissue and exposed to said first type of imaging said end members can be readily discerned in the targeted tissue, with said spacer being less discernable in the targeted tissue.

3. The fiducial marker of claim 1 wherein said textured outer surface is grooved.

4. The fiducial marker of claim 1 wherein each of said end members is of a cylindrical shape.

5. The fiducial marker of claim 1 wherein said sleeve is formed of a heat-shrinkable material that has been shrunk so that said end members are frictionally held at said positions by frictional engagement with said central passageway of said sleeve.

6. The fiducial marker of claim 4 wherein said sleeve is formed of a heat-shrinkable material that has been shrunk so that said end members are frictionally held at said positions by frictional engagement with said central passageway of said sleeve.

7. The fiducial marker of claim 1 wherein said sleeve is formed of an absorbable material.

8. The fiducial marker of claim 7 where said absorbable material comprises a dyed 90/10 PGA PLLA copolymer.

9. The fiducial marker of claim 1 wherein said spacer is formed of an absorbable material.

10. The fiducial marker of claim 9 wherein said absorbable material comprises and undyed 20/80 PLLA/PGA copolymer.

11. The fiducial marker of claim 7 wherein said spacer is formed of an absorbable material.

12. The fiducial marker of claim 9 wherein said absorbable material of said spacer comprises a dyed 90/10 PGA PLLA copolymer and wherein said absorbable material of said sleeve comprises and undyed 20/80 PLLA/PGA copolymer.

13. The fiducial marker of claim 1 wherein each of said end members comprises gold.

14. The fiducial marker of claim 12 wherein each of said end members comprises gold.

15. The fiducial marker of claim 1 wherein said spacer is substantially rigid whereupon the spacing between said end members of said marker is maintained at a fixed distance.

16. The fiducial marker of claim 4 wherein each of said end members has an outer diameter in the range of approximately 0.2 mm to 2 mm.

17. The fiducial marker of claim 16 wherein each of said end members has a length in the range of approximately 1 mm to 12 mm.

18. The fiducial marker of claim 17 wherein each of said end members has a center and wherein said spacer has a length so that the spacing between the centers of said end members is in the range of approximately 5 mm to 50 mm.

19. The fiducial marker of claim 17 wherein each of said end members has a center and wherein said spacer has a length so that the spacing between the centers of said end members is approximately 20 mm.

20. The fiducial marker of claim 5 wherein said sleeve has a thickness in the range of approximately 0.038 mm and 0.051 mm.

21. The fiducial marker of claim 1 wherein said fiducial marker has an over-all dumbbell-like shape, thereby rendering it further resistant to migration once implanted.
